(19) United States
(12) Patent Application Publication
(10) Pub. No.: US 2025/0375142 A1
(43) Pub. Date: Dec. 11, 2025

(54) MOTION-ROBUST CONTINUOUS HEART RATE MEASUREMENT

(71) Applicant: Verily Life Sciences LLC, Dallas, TX (US)

(72) Inventors: Nishant Verma, Cary, NC (US); Miles Bennett, San Francisco, CA (US); Charles Mathy, Arlington, MA (US)

(21) Appl. No.: 19/194,923

(22) Filed: Apr. 30, 2025

Related U.S. Application Data
(60) Provisional application No. 63/656,838, filed on Jun. 6, 2024.

(51) Int. Cl. A61B 5/355; A61B 5/024

(52) U.S. Cl. CPC A61B 5/355; A61B 5/02416; A61B 5/02438

(57) ABSTRACT

A technique of estimating a continuous heart rate of a subject includes acquiring pulsatility data indicative of a cardiac cycle of the subject and motion data indicative of a motion of the subject. Frequency transformations are performed on the pulsatility and motion data over discrete windows of temporal length T to generate a set of windowed spectra. The discrete windows are temporally overlapping and each temporally offset from one another. Heart rate values, each corresponding to one of the windowed spectra, are output using a processing pipeline including one or more neural networks trained to collectively output one of the heart rate values for each windowed spectra. The continuous heart rate is estimated based upon a set of the heart rate values from the discrete windows that are temporally overlapping.

### CROSS REFERENCE TO RELATED APPLICATION

This application claims priority to U.S. Provisional Application No. 63/656,838, filed on Jun. 6, 2024, the contents of which are incorporated herein by reference.

### TECHNICAL FIELD

This disclosure relates generally to heart rate measurement, and in particular but not exclusively, relates to continuous heart rate measurement using photoplethysmography.

### BACKGROUND INFORMATION

Photoplethysmography (PPG) is frequently used in wearable devices (such as smartwatches, fitness trackers, and smart rings) to measure blood volume changes at different parts of the body (such as a finger, the chest, or a wrist). With each heartbeat, the heart pumps blood through the cardiovascular system and arterial blood volume changes can be measured using PPG, where each cardiac cycle manifests as a peak in the PPG signal. This presents an opportunity to automatically analyze the PPG signal and obtain physiologically meaningful data such as heart rate, that can be used for both wellness as well as clinical monitoring purposes.

A significant challenge in designing methods to continuously measure heart rate is the high sensitivity of PPG signals to motion. FIG. 1A illustrates an example of a PPG signal affected by motion artifacts while a subject is moving and FIG. 1B illustrates PPG data acquired while the subject is at rest. As can be seen, the motion artifacts obscure the cardiac cycle. The presence of motion artifacts makes designing a robust algorithm to measure continuous heart rate a challenging problem.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A (Prior Art) illustrates an example photoplethysmography (PPG) signal with motion artifacts obscuring the cardiac cycle.

FIG. 1B (Prior Art) illustrates an example clean PPG signal without motion artifacts obscuring the cardiac cycle.

FIG. 2 illustrates a system for measuring/monitoring a continuous heart rate of a subject using a body wearable device, in accordance with an embodiment of the disclosure.

FIG. 3 is a flow chart illustrating a process for obtaining a motion-robust continuous heart rate measurement using PPG and motion data, in accordance with an embodiment of the disclosure.

FIG. 4 illustrates the windowing of raw data signals including pulsatility and motion data, in accordance with an embodiment of the disclosure.

FIG. 5 illustrates the preprocessing and frequency transformation of the pulsatility and motion data into windowed spectra of a spectrogram, in accordance with an embodiment of the disclosure.

FIG. 6 is a high-level functional block diagram illustrating the generation of heart rate values and confidence scores based upon the windowed spectra, in accordance with an embodiment of the disclosure.

FIG. 7 is a functional block diagram illustrating further details of the generation of heart rate values and confidence scores based upon the windowed spectra, in accordance with an embodiment of the disclosure.

FIG. 8A is a functional block diagram illustrating a first implementation of a processing pipeline for generating heart rate values from a sequence of windowed spectra, in accordance with an embodiment of the disclosure.

FIG. 8B is a functional block diagram illustrating a second implementation of a processing pipeline for generating heart rate values from a sequence of windowed spectra, in accordance with an embodiment of the disclosure.

### DETAILED DESCRIPTION

Embodiments of a system, apparatus, and method for estimating a continuous heart rate of a subject in a motion-robust manner are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

During daily living, a typical person engages in activities with varying levels of motion (such as exercise, walking, typing on a computer, etc.). Therefore, robustness to motion is important for a system capable of continuous measurement of heart rates. Even if a system (device and methods) is robust to motion artifacts, there are scenarios when motion can be too intense such that the captured pulsatility data (e.g., photoplethysmography signal) is almost completely corrupted by motion artifacts and it's nearly impossible to distinguish cardiac cycle peaks from peaks introduced by motion artifacts. In such scenarios, instead of returning an unreliable heart rate measure to a user, it can be more meaningful to return a heart rate measure with an associated confidence score. The confidence score should be high when the heart rate estimate is reliable and low when the heart rate estimate is less reliable. Confidence measures or scores can be difficult to estimate based on a snippet of pulsatility data, as it's challenging to distinguish between peaks generated by cardiac cycles versus peaks generated by motion artifacts. When the motion is random in nature (such as typing), the problem becomes even more challenging.

Conventional methods have been proposed for continuous heart rate measurement. Many of these methods were developed for electrocardiogram (ECG) based devices, which are typically worn around the chest location and are inherently less susceptible to motion artifacts than pulsatility signals measured using PPG. However, wearing a chest patch/strap in everyday life is cumbersome and often not practical. This has limited the usage of ECG patches for long-term, continuous monitoring of heart rates. Continuous measurement of heart rate using ECG has not been achieved at locations like the wrist, finger, ankle or other locations suitable for long-term continuous monitoring.

Some of these conventional methods have been adapted for heart rate measurement using PPG systems. However, they tend to be significantly affected by motion artifacts. When motion is present in the PPG signal, these conventional methods either (1) stop measuring heart rates and optionally return the last known reliable heart rate, or (2) return unreliable heart rates without any confidence measures associated with the data. As a user, it becomes challenging to interpret the heart rate data because the user can't tell which heart rates are reliable. In other words, the conventional techniques either can't provide a continuous heart rate measurement in the presence of significant motion artifacts or output the heart rate measurement with equal confidence irrelevant of motion artifacts.

The techniques described herein provide for continuous measurement/estimation of a heart rate that is robust to motion artifacts and is able to handle periods of vigorous user motion. The technique generates a confidence score associated with the estimated continuous heart rate, which reflects the reliability of the estimate. Heart rates estimated during periods of vigorous motion can then be considered by the end user or downstream algorithms with an appropriate level of suspicion or confidence.

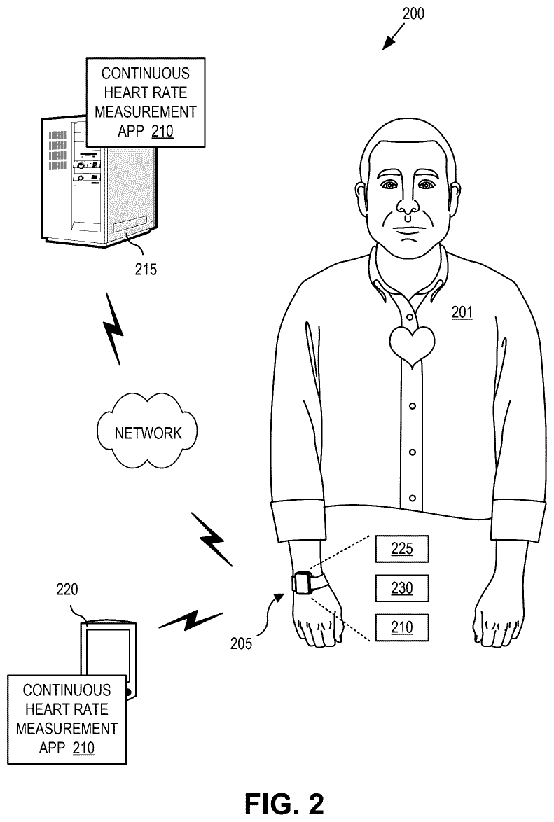

FIG. 2 illustrates a system 200 for measuring/monitoring a continuous heart rate of a subject 201 using a body wearable device 205, in accordance with an embodiment of the disclosure. System 200 includes body wearable device 205 along with one or more continuous heart rate measurement applications 210 installed either in the cloud on a cloud computing platform 215 or locally on an edge/personal computing platform 220 (e.g., smartphone, tablet, laptop, etc.). In one embodiment, a lightweight instance of continuous heart rate measurement application 210 may even be installed on body wearable device 205 itself. While body wearable device 205 is depicted having a wristwatch form factor, it should be appreciated that the techniques described herein are equally applicable to other form factors including a finger ring, an ankle bracelet, a neckband, an armband, or otherwise. The described methods are not specific to any anatomical location (such as wrist or finger) and can be used across locations where a cardiac cycle can be detected using PPG or other means of measurement. The accuracy of heart rate measurement may differ across anatomical locations because of susceptibility of the location to motion artifacts, environmental factors (such as temperature at a finger location), low tissue perfusion, etc.

Body wearable device 205 includes a pulsatility sensor 225 for acquiring pulsatility data indicative of a cardiac cycle of subject 201 and a motion sensor 230 for acquiring motion data indicative of a motion of subject 201 while the pulsatility data is measured. In one embodiment, pulsatility sensor 225 is a PPG sensor that measures and records PPG signals and motion sensor 230 is a three-axis accelerometer (e.g., orthogonal axes X, Y, and Z) that measures and records accelerations in three orthogonal dimensions. The pulsatility data and motion data is collectively referred to as the “raw signals” or “raw data.” The techniques described herein are not limited to just PPG data measured from a PPG sensor as it is anticipated that the techniques are equally applicable to other forms of pulsatility data. Regardless, the techniques are described with reference to PPG for convenience. For example, during operation the pulsatility data is sampled at sampling time tₙ, and specifically referred to as PPG(t) while the motion data is specifically referred to as X(t), Y(t), and Z(t). Furthermore, although body wearable device 205 is illustrated as a single wearable component, body wearable device 205 may be implemented as a multi-component system simultaneously worn on multiple dispersed body parts.

The logic that is executed to perform the techniques described herein is incorporated into continuous heart rate measurement application 210. As illustrated, continuous heart rate measurement application 210 may be installed in the cloud or locally on a personal processing apparatus. The logic of continuous heart rate measurement application 210 may even be installed and executed directly on body wearable device 205. Regardless, the pulsatility and motion data (or encoded versions thereof) may be buffered at body wearable device 205 for onboard processing and immediate display to subject 201, transmitted to computing platform 220 for local processing and display, or transmitted over a network (e.g., the Internet) for remote processing and monitoring at cloud computing platform 215.

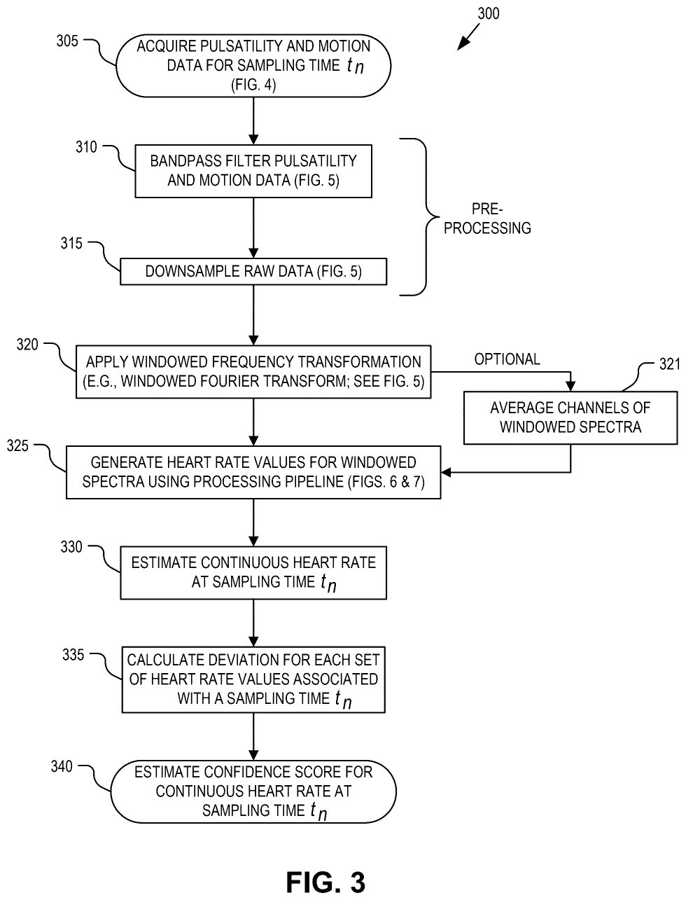

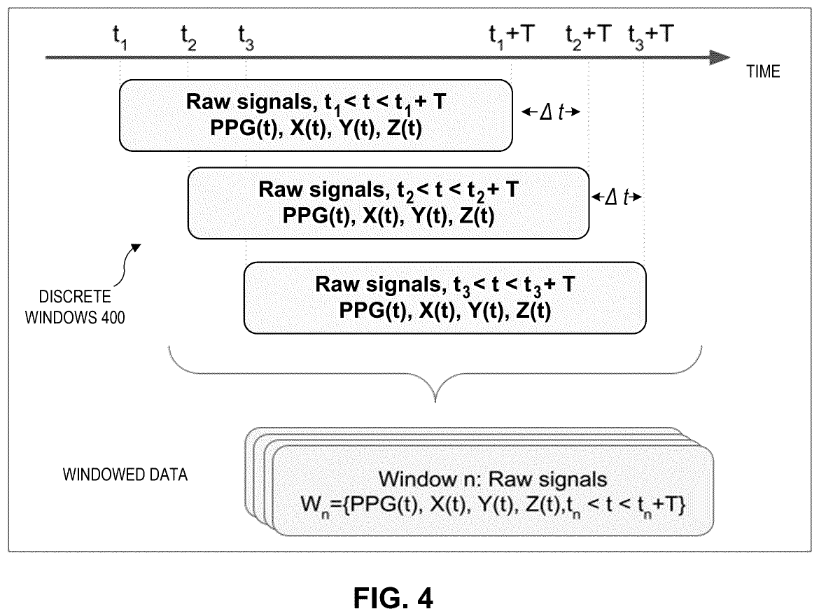

FIG. 3 is a flow chart illustrating a process 300 for obtaining a motion-robust continuous heart rate measurement, in accordance with an embodiment of the disclosure. Process 300 is described with reference to FIGS. 4-8. The order in which some or all of the process blocks appear in process 300 should not be deemed limiting. Rather, one of ordinary skill in the art having the benefit of the present disclosure will understand that some of the process blocks may be executed in a variety of orders not illustrated, or even in parallel.

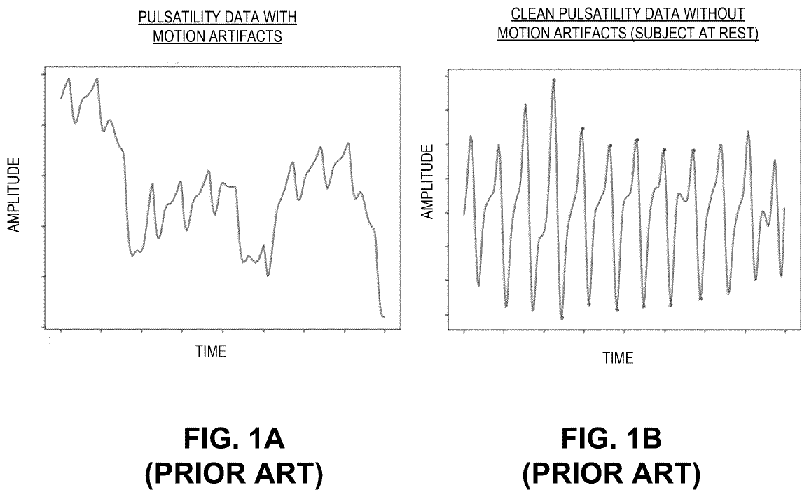

In a process block 305, the raw data is acquired by pulsatility sensor 225 and motion sensor 230 of body wearable device 205 and communicated to at least one instance of continuous heart rate measurement application 210. The raw data is measured at sampling time tₙ, where n is an integer. The raw data includes both the pulsatility data PPG(t) and the motion data X(t), Y(t), and Z(t) captured contemporaneously. Measuring heart rate during motion is challenging because motion artifacts manifest as peaks in the pulsatility data, which may be misinterpreted as cardiac cycles. Looking at the PPG data with motion artifacts (e.g., see FIG. 1A), it can be challenging to separate the heart rate signal from the motion-related signal. This is especially true if motion artifacts have overlapping frequency content with the heart rate data. However, since the motion data (along X, Y, and Z axes) captures information only related to motion, the PPG(t) and X(t), Y(t), and Z(t) signals can be analyzed together to provide a robust measurement of continuous heart rate.

The PPG and motion data from body wearable device 205 may have different sampling rates as well as signal characteristics (such as noise profiles and signal magnitude). If the PPG and motion data are visualized together in the time domain, it can be challenging to interpret the data together. Therefore, as a preliminary step, the raw data is preprocessed and transformed into a form that becomes more interpretable and easier for heart rate measurement.

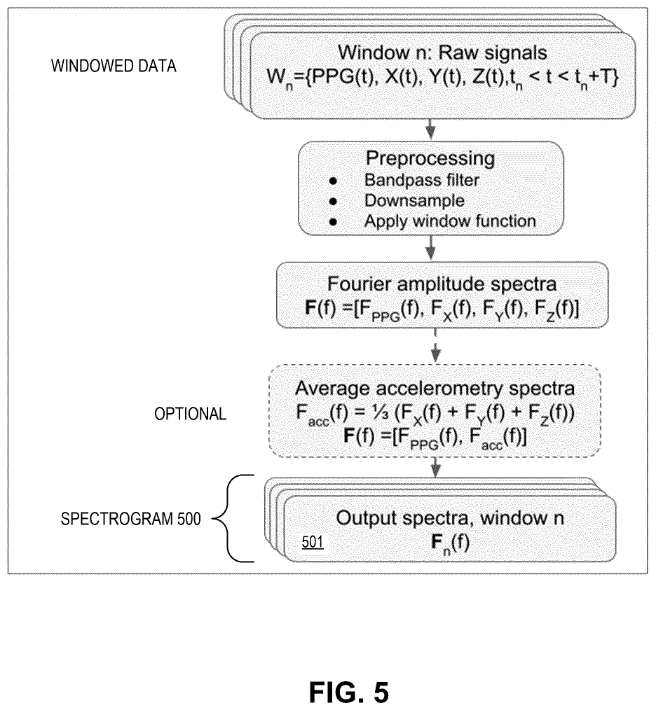

In the illustrated embodiment, the input PPG(t) data and motion data (X(t), Y(t), and Z(t)) are first converted to spectrograms 500 (FIG. 5), with each spectrogram 500 include a set of windowed spectra 501 originating from discrete windows 400 that are temporally offset and overlapping. In short, a spectrogram 500 corresponds to a time sequence of spectra 501 (e.g., Fourier spectra) that overlaps. A window of time T (temporal length or period T) is defined over which to compute the spectra, for example T=10 seconds. The discrete window of time T is then moved forward by some time interval Δt, for example Δt=1 second, and the Fourier spectra is computed again. FIGS. 4 and 5 illustrate the processing operations to obtain windowed spectra 501 (e.g., windowed Fourier spectra).

In a process block 310, before computing windowed spectra 500, the pulsatility data PPG(t) and motion data X(t), Y(t), and Z(t) are bandpass filtered (e.g., between 0.5 to 5 Hz) to retain heart rate-related information but discard deleterious components, such as baseline drifts in the PPG data and high frequency noise. The two sensor signals are then down-sampled to a common sampling rate (e.g., 30 Hz) that is sufficiently high to retain all heart rate-related information (process block 315).

In a process block 320, a frequency transformation (e.g., Fourier transform) is performed on the pulsatility and motion data over discrete windows 400. For example, Fourier spectra of the PPG(t), X(t), Y(t), and Z(t) data is calculated using a Fast Fourier Transform (FFT) with an applied windowing function (e.g., Hamming window). In other words, the Fourier spectra is an estimate of the power spectral density computed via the FFT. Optionally, the power spectral densities of the windowed spectra associated with each motion axis (i.e., motion data channels X, Y, and Z) may be averaged to generate orientation-invariant windowed spectra (process block 321). This is particularly useful when body wearable device 205 is a system distributed across several physically distinct wearable components, which may have different orientations for their accelerometer's X, Y, and Z axes. If there are multiple pulsatility data channels, they may be treated independently or their Fourier spectra can be averaged to increase the signal-to-noise ratio (SNR). This may also be useful when the pulsatility data is acquired across several physically distinct wearable components, which may each have a different number of pulsatility data channels.

Accordingly, for each discrete window 400 of temporal length T, a collection or set of windowed spectra 501 (e.g., windowed Fourier spectra) from the pulsatility and motion data channels is used to represent the sensor signals in that discrete window 400. The methods described below produce a heart rate value for each windowed spectra 501. It should be appreciated that while a set of windowed spectra 501 is represented herein as a spectrogram 500 for ease of interpretability, other representations of the signal data (such as Gabor wavelets, synchro-squeezed transforms, cepstra, etc.) may also be used to generate combined representations of the pulsatility and motion data. These representations are expected to produce similar performance of heart rate measurement.

The above preprocessing and windowed frequency transformation operations may be performed by a first software component/module of continuous heart rate measurement application 210, while the following operations of generating heart rate values and calculation of their associated confidence scores may be performed by their own respective components/modules. In one embodiment, heart rate values are estimated using a neural network that analyzes windowed spectra 501. This machine learning (ML) approach estimates a heart rate value for every discrete time window (i.e., each discrete window 400) of the PPG and motion data. For training the ML algorithm, ground truth data could be determined from a variety of sources. For example, ground truth labels could be obtained from electrocardiogram (ECG) signals, clinician labels, output from a simulator that generates synthetic data, or other types of heart rate monitors (e.g., ballistocardiogram). In the case of ECG training data, the ground truth heart rate for a discrete window 400 may be obtained by collecting the set of interbeat intervals (IBIs) in that time window using ECG. An IBI is the time interval from one heartbeat to the next in seconds and can be obtained by finding the R-R intervals (distance between consecutive R waves) in an ECG signal. The heart rate over that discrete window of data may then be defined as:

$$Heart Rate = 60 / mean ( IBIs ) ( bpm ) \qquad ( l )$$

Of course, other definitions for heart rate may also be used. For example, the mean can be replaced by the median to obtain a heart rate estimate that is more robust to outliers. To compute the ground truth heart rate over a time window, a specific time window duration (e.g., corresponding to temporal length T) must first be selected. A heart rate averaged over a large temporal period, for example a minute, will change more slowly than a heart rate averaged over a smaller temporal period. The methods described herein can be used for a heart rate averaged over any temporal length T. The heart rate averaged over a smaller time window is typically harder to estimate, because less signal is used for estimation, leading to estimates that have more noise and vary more quickly over time.

Each collection of windowed spectra 501 is associated with a discrete window 400 of data over which the Fourier spectra were computed (process block 320). This time window 400 has an associated ground truth heart rate, computed using equation (1) over the IBIs in the time window 400, or a similar equation. The task of heart rate estimation corresponds to estimating the heart rate for each window 400 of data. One possible approach is to treat each window independently, and try to estimate its heart rate directly. However, such a direct approach may suffer from several disadvantages. For example, if the data in a window happens to be noisy, or corrupted by noise, then the heart rate estimate may be unreliable. Another disadvantage is that using only a single window does not make use of known physiological constraints to heart rate. For example, heart rates have a typical rate of change—they do not jump by 30 bpm over one second. Treating each window independently does not incorporate knowledge about heart rate changes over time.

A common signal processing approach to including information from neighboring windows is to estimate a heart rate for each window independently, and run a tracking filter over the independent estimates. However, trackers suffer from several challenges. If a tracker encounters inaccurate estimates it may end up giving inaccurate estimates over a relatively large time window and get stuck in the poor estimate. Since inaccurate estimates can occur during motion, they may persist over a long enough time window to throw off the tracker. Conventional signal processing trackers typically either smoothen out the signal either too much, or too little, and do not have enough parameters to lead to reliable performance. If one can output a confidence estimate for each heart rate, then a tracking filter can incorporate this confidence, using confidence filtering. Intuitively, the tracking filter can change the amount to which it incorporates new heart rate estimates depending on the confidence.

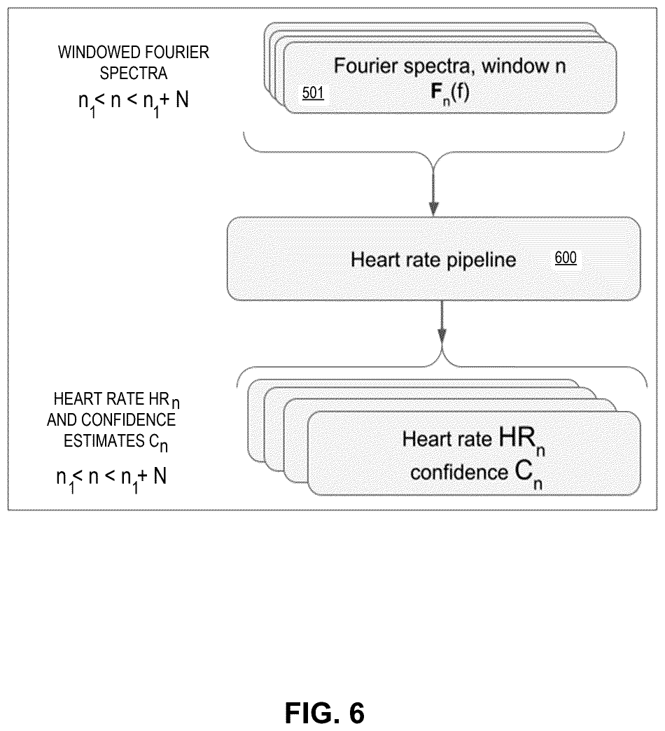

Accordingly, a processing pipeline 600 (also referred to as heart rate pipeline 600) that includes one or more neural networks trained to collectively output a heart rate value is used (see FIG. 6). Processing pipeline 600 takes windowed spectra 501 of a spectrogram 500 as input and outputs a heart rate value for each windowed spectra 501 (processing block 325). In the illustrated embodiments, processing pipeline 600 takes in a consecutive series of widowed spectra 501 of a spectrogram 500 and outputs heart rate values HRₙ as a series of consecutive heart rate values. The neural network(s) of processing pipeline 600 have access to a set of windowed spectra 501 and can therefore use contextual information to improve estimates. One can think of the neural networks as combining feature extraction with running a tracker with many more parameters than a typical signal processing tracker. Namely, because the neural networks output a series of heart rates directly, they can learn the contextual information of heart rates to improve estimates. For example, processing pipeline 600 can output 60 heart rates directly at a rate of one estimate per second. Note that the output frequency of heart rate estimation does not need to be equal to the input frequency of the windowed spectra 501. The neural networks of processing pipeline 600 can learn to output heart rates at any frequency.

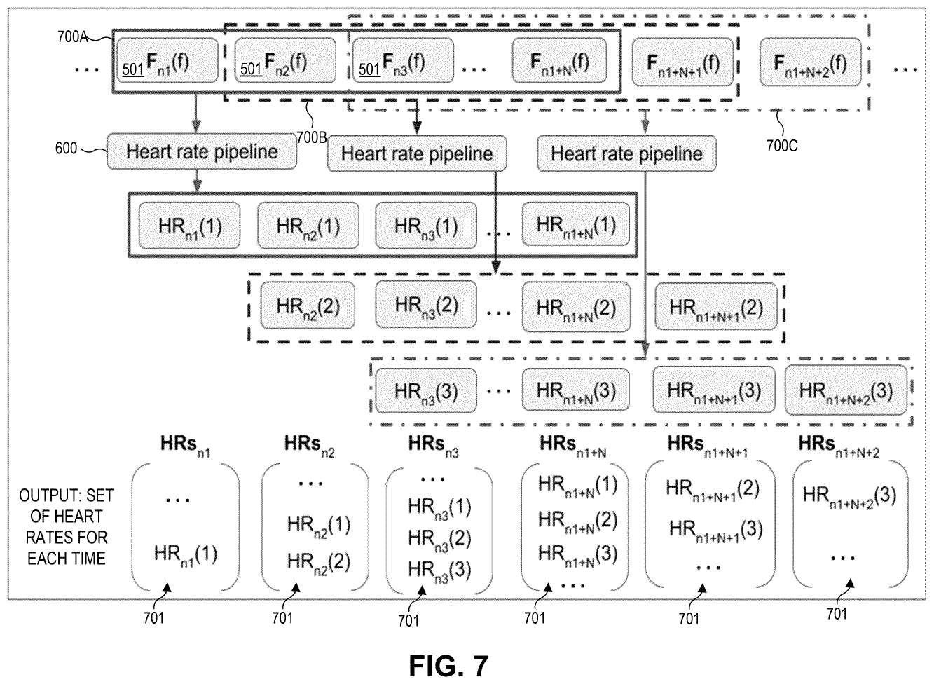

In process block 325, processing pipeline 600 runs on consecutive series of windowed spectra 501, with overlap (e.g., see FIG. 7, spectrograms 700A, B, C include overlapping windowed spectra 501). This leads to multiple heart rate values per time point and the generates sets 701 of heart rate values for each time t. A continuous heart rate can then be estimated for each time t based upon the set 701 of heart rate values 701 associated with time t (processing block 330). For example, the estimated continuous heart rate may be computed as an average or mean of the set 701.

Collecting a set 701 of heart rate values HRₙ for each time point t, further enables calculation of a deviation (e.g., standard deviation) for the set 701 at time t (process block 335) and estimation of a confidence score for each time t based on the calculated deviation (process block 340). The intuition here is that if a given heart rate value is estimated using only data before it, and then estimated using some data before it and some data after it, etc., then a set of heart rate values using different intervals of data around the estimation time point t may be computed. The more these heart rate values agree with each other in a given set 701, the more confidence in the continuous heart rate computed from that set 701. Accordingly, the confidence score for a given set 701 is inversely proportional to the standard deviation of the heart rate values for the given set 701. Namely, if the standard deviation is high, then the confidence score is low. Accordingly, in the illustrated embodiment, the output of processing pipeline 600 includes sets 701 of heart rate values for each time t from which confidence scores Cn and a continuous heart rate may be computed for each time t. In one embodiment, separate statistics sub-modules/components of continuous heart rate measurement application 210 may calculate the average and deviation of sets 701 to compute the continuous heart rate and confidence scores, respectively.

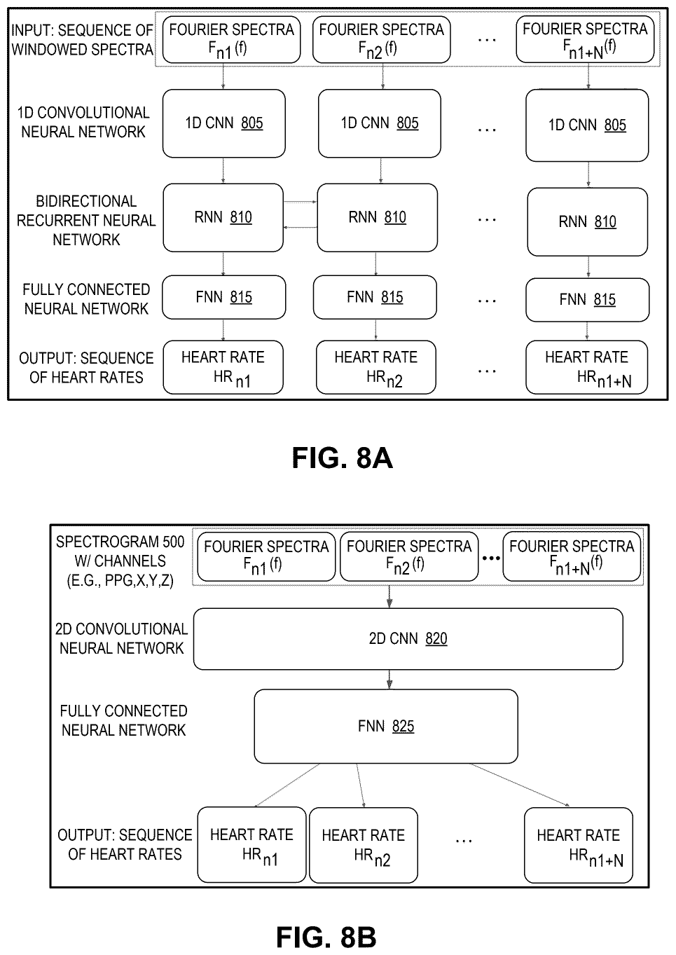

FIGS. 8A and 8B illustrate two example implementations for processing pipeline 600. FIG. 8A illustrates a first implementation including one-dimensional (1D) convolutional neural networks (CNNs) 805, followed by recurrent neural networks (RNNs) 810, followed by fully connected neural networks (FNNs) 815, which output the heart rate values HRₙ. In one embodiment, RNNs 810 may be replaced with Long Short-Term Memory (LSTM) modules or transformer modules. Batch normalization and/or dropout layers may be inserted between each neural network layer to improve regularization. FIG. 8B illustrates a second implementation including a single two-dimensional (2D) CNN 820 followed by a FNN 825, which outputs a series of heart rate values HRₙ. Treating the consecutive series of windowed spectra 501 as a spectrogram 500 (i.e., an image), 2D CNN 820 can receive spectrogram 500 as a whole. The neural networks of either implementation of processing pipeline 600 can be trained by having participants wear a chest ECG to acquire ground truth data associated with the windowed spectra 501 and spectrograms 500.

Once the neural networks of processing pipeline 600 have been trained to output a consecutive series of heart rate values (process block 325), an estimate of confidence is obtained via the approach depicted in FIG. 7 (processing blocks 335 & 340). The heart rate pipeline is run on a consecutive series of windowed spectra 501, outputting a series of heart rate values. Instead of moving onto the next batch of windowed spectra 501 with no overlap, heart rate pipeline 600 is run again on a set of windowed spectra 501 that includes some of the windowed spectra 501 it just ran on. For example, if heart rate pipeline 600 ran on windowed spectra 1 through 60, then it is run again on spectra 2 through 61, and again on spectra 3 through 62, etc. In doing so, a redundant set of heart rate values for each time t is obtained. This redundant set carries a wealth of useful information. Intuitively, if for example heart rate values are estimated at time point t=60 using windowed spectra 1 through 60, and then again using windowed spectra 60 through 119, then the calculated heart rate values are using different context windows—the former one is only using data in the past and the latter one is only using data in the future. Collecting sets 701 of heart rate values using different amounts of data before and after the estimation time point t, we obtain sets of heart rate values that intrinsically carry information about the reliability of the estimates. For example, if all heart rate values of a set 701 agree, that gives more confidence about the continuous heart rate, which is computed as an average of the heart rate values HRₙ in the given set 701. On the other hand, if the heart rate values HRₙ of a given set 701 vary wildly, then the continuous heart rate computed as an average of these wildly varying heart rate values HRₙ, may be unreliable.

One way to obtain a confidence score from a set 701 of heart rate values at each time point t is to compute the following quantity:

$$c = {e}^{- {α}_{0} {σ}_{{HR}^{2}}} , \qquad ( 2 )$$

where c represents a confidence score, σ_{HR} represents a standard deviation of the heart rate values for a given time t, and α₀ is a scaling factor for the confidence score (α₀>0). This number will be high when confidence is high, as the standard deviation will be low. Conversely, if the standard deviation is high, then the confidence will be low. The above estimate of confidence has a convenient interpretation because it is bounded between 0 and 1. A value of 1 occurs when the standard deviation is 0, and a value of 0 occurs in the limit as standard deviation approaches infinity. The estimate may be normalized by changing the value of do, or instead of standard deviation, one could use a more outlier robust estimate like mean absolute deviation from the mean or median absolute deviation. It should be appreciated that numerous other methods leveraging the similarity or variability between the heart rate values calculated at each time point t may be implemented in place of a standard deviation computation.

The continuous heart rate may be estimated at each time point t (process block 330) using mean, median, quantiles (such as 25% or 75%), or other aggregate statistics (such as outlier-robust mean) from the vector representing a set 701. Due to its robustness to outliers, the median heart rate value may represent an appropriate estimate for continuous heart rate. Another approach is to report the continuous heart rate from the series of windowed spectra 501 that is centered at the current time point (i.e., the neural networks have equal amounts of context before and after the current time point where a heart rate value is being computed).

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A method of estimating a continuous heart rate of a subject, the method comprising:
acquiring pulsatility data indicative of a cardiac cycle of the subject during sampling time tₙ, where n is an integer;
acquiring motion data indicative of a motion of the subject while the pulsatility data is measured from the subject during the sampling time tₙ;
performing frequency transformations on the pulsatility and motion data over discrete windows of a temporal length T to generate a set of windowed spectra, wherein the discrete windows are temporally overlapping and each temporally offset from one another;
outputting heart rate values each corresponding to one of the windowed spectra using a processing pipeline including one or more neural networks trained to collectively output one of the heart rate values for each of the windowed spectra; and
estimating the continuous heart rate for the sampling time tₙ based upon a set of the heart rate values associated with the sampling time tₙ from the discrete windows that are temporally overlapping.

2. The method of claim 1, further comprising:
calculating a deviation for the set of heart rate values associated with the sampling time tₙ; and
estimating a confidence score for the continuous heart rate at the sampling time tₙ based on the deviation.

3. The method of claim 1, wherein each of the discrete windows is temporally offset from an adjacent one of the discrete windows by a time increment Δt, where Δt<T.

4. The method of claim 1, wherein the frequency transformations comprise windowed Fourier transforms and the windowed spectra comprise windowed Fourier spectra.

5. The method of claim 1, wherein the pulsatility data comprises photoplethysmography (PPG) data and the motion data comprises accelerometry data.

6. The method of claim 5, wherein the PPG and accelerometry data are acquired from at least one body wearable device.

7. The method of claim 5, wherein the accelerometry data comprises three-axis accelerometry data, the method further comprising:
averaging power spectral densities of the windowed spectra associated with each axis of the three-axis accelerometry data to generate orientation-invariant windowed spectra.

8. The method of claim 1 further comprising:
bandpass filtering the pulsatility and motion data prior to performing the frequency transformations.

9. The method of claim 1, wherein the processing pipeline comprises:
one-dimensional (1D) convolutional neural networks (CNNs) each associated with one of the windowed spectra within the set of windowed spectra;
one of bi-directional recurrent neural networks (RNNs), long short-term memory (LSTM), or transformers each coupled to an output of an associated one of the 1D CNNs; and
fully connected neural networks (FNNs) each coupled to an output of an associated one of the RNNs, LSTM, or transformers, wherein the FNNs are trained to output the heart rate values.

10. The method of claim 1, wherein the processing pipeline comprises:
a two-dimensional (2D) convolutional neural network (CNN) with inputs adapted to receive the set of windowed spectra; and
a fully connected neural network (FNN) coupled to an output of the 2D CNN and trained to output the heart rate values.

11. At least one machine-readable medium having instructions stored thereon that, in response to execution, cause a computing system to perform operations for estimating a continuous heart rate of a subject, the operations comprising:
acquiring pulsatility data indicative of a cardiac cycle of the subject during sampling time tₙ, where n is an integer;
acquiring motion data indicative of a motion of the subject while the pulsatility data is measured from the subject during the sampling time tₙ;
performing frequency transformations on the pulsatility and motion data over discrete windows of a temporal length T to generate a set of windowed spectra, wherein the discrete windows are temporally overlapping and each temporally offset from one another;
outputting heart rate values each corresponding to one of the windowed spectra using a processing pipeline including one or more neural networks trained to collectively output one of the heart rate values for each windowed spectra; and
estimating the continuous heart rate for the sampling time tₙ based upon a set of the heart rate values associated with the sampling time tₙ from the discrete windows that are temporally overlapping.

12. The at least one machine-readable medium of claim 11, the operations further comprising:
calculating a deviation for the set of heart rate values associated with the sampling time tₙ; and
estimating a confidence score for the continuous heart rate at the sampling time tₙ based on the deviation.

13. The at least one machine-readable medium of claim 11, wherein each of the discrete windows is temporally offset from an adjacent one of the discrete windows by a time increment Δt, where Δt<T.

14. The at least one machine-readable medium of claim 11, wherein the frequency transformations comprise windowed Fourier transforms and the windowed spectra comprise windowed Fourier spectra.

15. The at least one machine-readable medium of claim 11, wherein the pulsatility data comprises photoplethysmography (PPG) data and the motion data comprises accelerometry data.

16. The at least one machine-readable medium of claim 15, wherein the PPG and accelerometry data are acquired from a body wearable device.

17. The at least one machine-readable medium of claim 15, wherein the accelerometry data comprises three-axis accelerometry data, the operations further comprising:
averaging power spectral densities of the windowed spectra associated with each axis of the three-axis accelerometry data to generate orientation-invariant windowed spectra.

18. The at least one machine-readable medium of claim 11, the operations further comprising:
bandpass filtering the pulsatility and motion data prior to performing the frequency transformations.

19. The at least one machine-readable medium of claim 11, wherein the processing pipeline comprises:
one-dimensional (1D) convolutional neural networks (CNNs) each associated with one of the windowed spectra within the set of windowed spectra;
one of bi-directional recurrent neural networks (RNNs), long short-term memory (LSTM), or transformers each coupled to an output of an associated one of the 1D CNNs; and
fully connected neural networks (FNNs) each coupled to an output of an associated one of the RNNs, LSTM, or transformers, wherein the FNNs are trained to output the heart rate values.

20. The at least one machine-readable medium of claim 11, wherein the processing pipeline comprises:
a two-dimensional (2D) convolutional neural network (CNN) with inputs adapted to receive the set of windowed spectra; and
a fully connected neural network (FNN) coupled to an output of the 2D CNN and trained to output the heart rate values.
